(19) United States
(12) Patent Application Publication
(10) Pub. No.: US 2024/0131219 A1
(43) Pub. Date: Apr. 25, 2024

(54) RADIOACTIVE GLASS MICROSPHERES FOR EMBOLIZATION, PREPARATION METHOD AND APPLICATION THEREOF

(71) Applicant: Sun Yat-sen University Cancer Center (The Affiliated Cancer Hospital of Sun Yat-sen University,, Guangdong (CN)

(72) Inventors: Sheng PENG, Guangdong (CN); Fujun ZHANG, Guangdong (CN); Ligong LU, Guangdong (CN); Dafeng YANG, Guangdong (CN)

(21) Appl. No.: 18/455,233

(22) Filed: Aug. 23, 2023

(30) Foreign Application Priority Data
Oct. 20, 2022 (CN) 2022112994652

(51) Int. Cl. A61L 24/02; A61L 24/00

(52) U.S. Cl. CPC A61L 24/02; A61L 24/0015; A61L 2300/44; A61L 2400/04

(57) ABSTRACT

The present invention provides radioactive glass microspheres for embolization and a preparation method and an application thereof. A nuclide oxide and a foaming agent are added into a glass matrix, blended and uniformly mixed for making the foaming agent decomposed and vaporized at a high temperature to generate bubbles, so as to prepare the radioactive glass microspheres for embolization with cavities. The radioactive glass microspheres for embolization have a density of 1.4-2.3 g/cm³, a nuclide loading rate of 15-40 wt % and a higher and more stable radiation dose, can achieve better distribution and deposition effects in liver blood vessels after injection, and can achieve a better therapeutic effect for hepatocellular carcinoma (HCC).

### CROSS-REFERENCE TO RELATED APPLICATIONS

The present invention claims the priority of a Chinese prior application No. 202211299465.2 filed on Oct. 21, 2022, the specification, claims and attached drawings of which are incorporated herein as a part of references of the present invention, and the present invention is also a partial continuing application based on the application.

### BACKGROUND OF THE INVENTION

### Field of the Invention

The present invention belongs to the technical field of biomedicine, and relates to radioactive glass microspheres for embolization, a preparation method and an application thereof.

### Description of the Related Art

During therapy for liver cancer, as normal hepatic cells have a lower radiation dose to be tolerated and hepatoma carcinoma cells required to be killed have a relatively higher radiation dose, external radiotherapy for the liver cancer has not been widely used in clinical practice.

However, during internal radiotherapy for the liver cancer, a radioactive embolization method can achieve the effects of relatively higher therapeutic effect and relatively fewer side effects. Specifically, radioactive embolization refers to a process of injecting radionuclide microspheres into hepatic arteries. The microspheres can be highly concentrated and retained in micro-blood vessels of the hepatoma carcinoma cells, so as to embolize nourishing blood vessels of tumors and kill surrounding carcinoma cells through radiation.

Radionuclide emitting rays loaded by the microspheres generally include a beta-ray and have an action distance within a few millimeters to tens of millimeters, which can cause the death of surrounding tumor cells and have little damage to normal hepatic cells far from the microspheres.

At present, two kinds of radioactive microspheres have been put into clinical use, including TheraSphere® microspheres developed by NORDION in Canada and SirSpheres® microspheres developed by Sirtex Medical in Australia. The two kinds of microspheres use a beta-ray emitted by yttrium-90 to achieve a therapeutic effect, but have different physical properties and production methods. The TheraSphere® microspheres are glass microspheres containing non-radioactive yttrium-89, and the yttrium-89 contained in the glass microspheres are activated into radioactive yttrium-90 by neutron activation before use (U.S. Pat. Nos. 4,789,501 and 5,011,677). The SirSpheres® microspheres use ion-exchange resin microspheres to adsorb activated yttrium-90 ions and cure the same in a phosphate form at adsorption sites (US20070253898A1). Glass microspheres are loaded with more radionuclides than resin adsorption microspheres. For example, each TheraSphere® glass microsphere has a radiation dose of about 2,500 Bq and a nuclide loading capacity of about 30%, while each SirSpheres® resin microsphere has a radiation dose of about 75 Bq. As for the same therapeutic dose, more resin microspheres are required. However, the glass microspheres have a higher density. The TheraSphere® glass microspheres have a density of 3.6 g/cm³, which is three times higher than the density of blood (1.05 g/cm³). Due to the too high density, the glass microspheres have a too high sedimentation velocity and poor distribution and deposition effects in liver blood vessels. The SirSpheres® microspheres have a density of 1.6 g/cm³, which is similar to the density of blood, thus having good distribution and deposition effects in liver blood vessels. However, such microspheres have a low nuclide loading capacity. According to SIRTEX, a method for preparing low-density radioactive glass microspheres for embolization is disclosed (U.S. Pat. No. 6,998,105), and the density can be as low as 2.2 g/cm³. With respect to such microspheres, the density of the microspheres is decreased by reducing the proportion of a high-density nuclide oxide in a glass matrix (only 2%). Although the density is decreased, the nuclide loading rate is much lower than that of the TheraSphere® microspheres (with a nuclide loading rate of 30%). Apart from glass, resins and other materials, degradable polymers (such as PLGA, PLLA and the like) are also used for preparing radioactive microspheres for embolization with lower density, while the stability and the nuclide loading rate are inferior to glass micropsheres.

According to U.S. Ser. No. 10/940,219B2, radioactive microspheres for radiation therapy are disclosed. Resin glass with nanopores is used for preparation, a solution containing a radionuclide is loaded into the nanopores on the surfaces of microspheres, the solution is evaporated until the radionuclide precipitates onto the surfaces of the nanopores in the microspheres, and then baking at high temperature is performed for fixing to obtain the radioactive microspheres. However, the radioactive microspheres prepared by this method still belong to resin microspheres and still have a low nuclide loading capacity, and as for the same therapeutic dose, more microspheres are required.

At present, radioactive microspheres that have a high nuclide loading capacity and a suitable density to achieve good distribution and deposition effects during injection are not provided.

### BRIEF SUMMARY OF THE INVENTION

In order to overcome the defects of the prior art, the present invention provides novel radioactive glass microspheres for embolization. A nuclide oxide and a foaming agent are added into a glass matrix, blended and uniformly mixed for making the foaming agent decomposed and vaporized at a high temperature to generate bubbles, so as to prepare the radioactive glass microspheres for embolization with cavities. The radioactive glass microspheres for embolization have a density of 1.4-2.3 g/cm³, a nuclide loading rate of 15-40 wt % and a higher and more stable radiation dose. It can achieve better distribution and deposition effects in liver blood vessels after injection, and can achieve a better therapeutic effect for liver cancer or hepatocellular carcinoma (HCC).

The cavities in the glass microspheres are formed during machining rather than being etched from the outside, so as to form microchannels communicated the outside. At a high temperature, the glass microspheres are melted into a liquid state, and gases released by decomposition of the foaming agent, such as carbon dioxide emitted from the microspheres, form bubbles in the melted glass microspheres. As the temperature is decreased rapidly, melted glass is cured rapidly, and the internal bubbles form cavities to obtain non-solid microspheres, so that the density of the microspheres is decreased. That is so say, the weight is decreased while the overall volume is unchanged, thereby decreasing the density.

On the one hand, the present invention provides radioactive glass microspheres for embolization. The glass microspheres include glass microsphere bodies and cavities formed in the glass microsphere bodies, and the glass microsphere bodies contain a nuclide oxide; and the glass microspheres have a density of not higher than 2.3 g/cm³.

Further, the nuclide oxide is any one or more of Y₂O₃, Lu₂O₃, Ho₂O₃ or P₂O₅; and the glass microsphere bodies further contain any one or more of Al₂O₃, SiO₂ and B₂O₃.

Further, the glass microspheres have a density of 1.4-2.3 g/cm³ and a nuclide loading rate of 15-40 wt %.

Due to a higher density, existing solid glass microspheres are quickly sedimentated to every corner of the bottoms of blood vessels after each injection and are difficult to be accurately located for vascular embolization, thus having a greatly reduced therapeutic effect.

In the present invention, the glass microspheres are prepared by adding a foaming agent into a glass matrix. As the foaming agent is decomposed at a high temperature to generate bubbles, the microspheres are more loose in texture and have more cavities, thus having a lower density. The microspheres are not immediately sedimentated to the bottoms of blood vessels after injection, but can be uniformly distributed on upper sides, lower sides, left sides and right sides of accurate positions in the blood vessels. Therefore, the microspheres have better distribution and deposition effects in liver blood vessels and can accurately accumulate in sites requiring vascular embolization to achieve a better vascular embolization effect.

The glass microspheres prepared by the present invention can achieve a high nuclide loading capacity and a higher radiation dose, is more suitable for being used as radioactive glass microspheres for embolization, and can achieve a better therapeutic effect. It is proven through research that compared with solid stripped microspheres without addition of a foaming agent, the glass microspheres prepared after foaming with a foaming agent are loose in texture and contain more cavities. A nuclide in the glass microspheres prepared therefrom can produce a higher radiation dose after neutron activation. The reason may be that a beta-ray emitted by yttrium-90 is more likely to radiate from a loose glass matrix with cavities and can produce a higher radiation dose and kill surrounding tumor cells more effectively, thereby having a better therapeutic effect.

The radiation dose of the present invention refers to an irradiation effect produced by each glass microsphere on the inside of a quantitative irradiated substance. For example, when an irradiation quantity generated by irradiation of 1 g of the radioactive glass microspheres is 2,500 Bq, the radiation dose is 2,500 Bq. The radiation dose may also be considered as a radiation intensity emitted by the microspheres of a same weight. It may be understood that as for the radiation or irradiation intensity, the irradiation intensity will be decreased or weakened when the microspheres pass through some barriers, such as plastics, steel plates and the like. However, the microspheres of the present invention have many pores, and a beta-ray emitted by a substance (yttrium-90) in the microspheres basically realizes irradiation or radiation directly without barriers and is basically not weakened, so that the irradiation intensity is generally enhanced compared with that of solid microspheres.

Further, the glass microspheres contain, by molar percentage, 0-40% of Al₂O₃, 20-80% of SiO₂, 0-20% of B₂O₃ and 10-30% of a nuclide oxide, and have a particle size of 10-100 μm.

Through optimization of a formula of the glass microspheres in the present invention, it has found that the glass microspheres with cavities containing B₂O₃ have a higher nuclide loading rate and an obviously higher radiation dose. After analysis of the microspheres, the reason may be that as the glass microspheres containing B₂O₃ have better properties, convenience is provided for foaming of the foaming agent to further improve the texture and fix more nuclide oxides, and radiation of a 3-ray is more facilitated to achieve a higher sustained radiation dose under the condition of a same nuclide loading capacity.

On the other hand, the present invention provides a method for preparing radioactive glass microspheres for embolization. The method includes the following steps:
(1) preparing glass microsphere bodies, mixing the glass microsphere bodies with a foaming agent to obtain a mixture, and melting the mixture by heating to obtain a glass matrix;
(2) cooling the glass matrix, followed by curing to form a glass block, and then grinding the glass block to obtain glass particles; and
(3) melting the glass particles by heating to obtain glass microspheres, and decomposing the foaming agent in the glass microspheres to generate gases so as to form cavities in the glass microspheres;
or the method includes the following steps:
(a) preparing glass microsphere bodies, and melting the glass microsphere bodies by heating to obtain a glass matrix;
(b) cooling the glass matrix, followed by curing to form a glass block, adding a foaming agent for mixing, and then performing grinding to obtain glass particles so as to make the foaming agent adsorbed on the surfaces of the glass particles; and
(c) melting the glass particles by heating, and decomposing the adsorbed foaming agent to generate gases so as to form cavities in the glass microspheres.

In the present invention, as a foaming agent is added into a glass matrix and the foaming agent is decomposed and vaporized at a high temperature during preparation of the glass microspheres, the glass microspheres are more loose in texture and have an obviously decreased density. Meanwhile, many hollow cavities are generated in the glass microspheres, which can not only further decrease the density, but also increase the nuclide loading rate and the radiation dose of the glass microspheres.

Meanwhile, in the radioactive glass microspheres for embolization provided by the present invention, as a nuclide oxide is directly added into a glass matrix, the nuclide oxide is more uniformly distributed in the glass matrix and has a better fusion effect with other components in the glass matrix during preparation of the microspheres. The microspheres have a high nuclide loading rate, a higher radiation dose in the process of emitting a β-ray to a disease site, and a longer stabilization time.

Further, the glass microsphere bodies further contain any one or more of Al₂O₃, SiO₂ and B₂O₃, and a nuclide oxide; and the nuclide oxide is any one or more of Y₂O₃, Lu₂O₃, Ho₂O₃ or P₂O₅.

Further, the foaming agent in step (1) and/or step (b) includes any one or more of a sulfate, a carbonate, an inorganic salt of a gas generated by high-temperature decomposition, and an organic polymer.

In some embodiments, the foaming agent includes any one or more of Na₂SO₄, MgSO₄, Na₂CO₃, CaSO₄, K₂CO₃, Li₂CO₃, SrCO₃, an inorganic salt of a gas produced by high-temperature decomposition, polyethylene glycol and polyvinyl alcohol.

Further, during the melting by heating in step (1) and/or step (a), the heating is performed at a temperature of 1,000-1,600° C.; and during the melting by heating in step (3) and/or step (c), the heating is performed at a temperature of 1,600-1,800° C.

The heating is performed at a temperature of 1,000-1,600° C. in step (1) and/or step (a), which is lower than the temperature of the foaming agent as much as possible, and the heating is performed to melt components of the glass matrix and promote uniform mixing after melting. In some embodiments, in order to prevent a large quantity of the foaming agent from decomposition in advance, other components may be heated, dissolved and mixed uniformly first, and finally the foaming agent is added, stirred and mixed uniformly.

The melting by heating in step (3) and/or step (c) is performed for decomposing the foaming agent to generate bubbles. Different foaming agents have different foaming temperatures, which generally start to decompose and generate bubbles at a temperature higher than 800° C. or 1,300° C.

Further, the method further includes a step (4) and/or a step (d): screening glass microspheres with a suitable particle size of 10-100 μm and a density of 1.4-2.3 g/cm³.

Further, the method for preparing radioactive glass microspheres for embolization provided by the present invention includes the following steps:
(1) mixing at least one of an Al₂O₃ powder, a SiO₂ powder and a B₂O₃ powder, as well as a nuclide oxide powder including or excluding a foaming agent, followed by full melting and mixing at 1,000-1,600° C. to prepare a uniform glass matrix; wherein in order to prevent a large quantity of the foaming agent from decomposition in advance, the foaming agent can be added after other components are completely melted and mixed uniformly;
(2) cooling the glass matrix obtained in step (1), followed by curing to form a glass block, and then grinding the glass block to obtain glass particles; wherein when the foaming agent is not added in step (1), the foaming agent is added in a solid or solution form and mixed and ground with the glass matrix in this step, so as to make the foaming agent adsorbed on the surfaces of the glass particles;
(3) melting the irregular glass particles obtained in step (2) at 1,600-1,800° C. to form glass microspheres, and decomposing the foaming agent in or adsorbed on the glass microspheres to generate gases so as to form cavities in the glass microspheres;
(4) cooling the glass microspheres obtained in step (3), curing the glass microspheres, and recycling the glass microspheres;
(5) screening glass microspheres with a suitable particle size, and screening glass microspheres with internal cavities that meet a density requirement; and
(6) performing neutron activation on a nuclide embedded in the glass microspheres obtained in step (5) to obtain the radioactive glass microspheres for embolization.

Further, the foaming agent in step (1) includes Na₂SO₄, CaSO₄, MgSO₄ and other sulfates, Na₂CO₃, K₂CO₃, Li₂CO₃ and other carbonates, and other inorganic salts of a gas produced by high-temperature decomposition, or polyethylene glycol, polyvinyl alcohol and other organic polymers.

Further, the irregular glass particles in step (2) have a particle size of 10-100 μm.

Further, the suitable particle size in step (5) is 10-100 μm, and the density is required to be 1.4-2.3 g/cm³.

Further, a solvent used for density screening includes electronic fluorinated liquids having different densities (such as 3M™Novec™HFE7100, 3M™Novec™ FC40, 3M™Novec™ FC70 and the like), 1,2-dibromoethane, an iopamidol solution and the like.

On another hand, the present invention provides use of the glass microspheres described above, or glass microspheres prepared by the method described above in preparation of radioactive glass microspheres for embolization that can be uniformly distributed and deposited at an injection site and can increase the radiation dose.

Further, the radioactive glass microspheres for embolization can increase the radiation dose of a β-ray.

Further, the radioactive glass microspheres for embolization contain, by molar percentage, 0-40% of Al₂O₃, 20-80% of SiO₂, 0-20% of B₂O₃ and 10-30% of a nuclide oxide.

On another hand, the present invention provides use of the radioactive glass microspheres for embolization in preparation of drugs for treatment of tumors.

In some embodiments, the tumors include hepatocellular carcinoma.

The present invention has the following beneficial effects.
1. As a foaming agent is decomposed and vaporized at a high temperature, the radioactive glass microspheres for embolization are more loose in texture and have large quantities of bubbles and cavities generated, thus having an obviously decreased density. Therefore, the radioactive glass microspheres for embolization have better distribution and deposition effects during injection and can accurately accumulate in sites requiring vascular embolization to achieve a better vascular embolization effect.
2. As a nuclide oxide is directly added into a glass matrix, the nuclide oxide is more uniformly distributed in the glass matrix and has a better fusion effect with other components in the glass matrix during preparation of the microspheres. The microspheres have a high nuclide loading rate, a higher radiation dose in the process of emitting a β-ray to a disease site, and a longer stabilization time.
3. The glass microspheres prepared after foaming with a foaming agent are loose in texture and contain more bubbles and cavities. After neutron activation of a nuclide in the glass microspheres, a beta-ray emitted by yttrium-90 is more likely to radiate from a loose glass matrix with cavities and can produce a higher radiation dose and kill surrounding tumor cells more effectively, thereby having a better therapeutic effect.
4. As B₂O₃ is required to be added in a glass matrix formula, comprehensive performance of the radioactive glass microspheres for embolization is further improved.
5. The preparation method is simple and efficient, the prepared radioactive glass microspheres for embolization are more balanced and stable, and clinical needs are met.

### BRIEF DESCRIPTION OF THE DRAWINGS

FIG. 1 shows optical microscope photographs (50 μm) of solid glass microspheres (A) and hollow glass microspheres (B) in Example 1.

FIG. 2 shows scanning electron microscope photographs (50 μm) of solid glass microspheres (A) and hollow glass microspheres (B) in Example 1.

FIG. 3 shows an optical microscope image (A) (50 μm) and a scanning electron microscope photograph (B) (20 μm) of glass microspheres for embolization obtained after density screening in Example 2.

FIG. 4 shows comparison of sedimentation effects of hollow microspheres and solid microspheres in a PBS solution at different time points in Example 3, wherein the left figure shows the sedimentation effect (A) when t is 0 second, and the right figure shows the sedimentation effect (B) when t is 1 minute.

FIG. 5A to FIG. 5E are images showing vascular embolization effects of solid microspheres, in which FIG. 5A is a distribution and angiography image of overall blood vessels before vascular embolization; FIG. 5B is an angiography image of blood vessels in an embolization area (without solid microspheres); FIG. 5C is an angiography image of overall blood vessels after vascular embolization;

FIG. 5D is a locally enlarged image of an angiography image of blood vessels in an embolization area before embolization (in a white dotted line box of FIG. 5B); and FIG. 5E is a locally enlarged image of blood vessels after embolization (in a white dotted box of FIG. 5C).

FIG. 6A to FIG. 6E are images showing vascular embolization effects of hollow microspheres of the present invention, in which FIG. 6A is a distribution and angiography image of overall blood vessels before vascular embolization; FIG. 6B is an angiography image of blood vessels in an embolization area (without solid microspheres); FIG. 6C is an angiography image of overall blood vessels after vascular embolization; FIG. 6D is a locally enlarged image of an angiography image of blood vessels in an embolization area before embolization (in a white dotted line box of FIG. 6B); and FIG. 6E is a locally enlarged image of blood vessels after embolization (in a white dotted box of FIG. 6C).

### DETAILED DESCRIPTION OF THE INVENTION

The present invention is described in further detail below in conjunction with examples. It is to be noted that the examples below are intended to facilitate understanding of the present invention, rather than to limit the present invention in any manner. Reagents not specifically identified in the examples are known products and are obtained by purchasing commercially available products.

### Example 1 Preparation of Radioactive Glass Microspheres for Embolization Provided by the Present Invention

A method for preparing radioactive glass microspheres for embolization is provided in this example. The method includes the following steps.

Certain quantities of Y₂O₃, SiO₂, Al₂O₃ and B₂O₃ were weighed, placed in a quartz grinder (specific proportions are as shown in Table 1) and fully ground for 10 minutes for uniform mixing. A uniformly mixed microsphere raw material powder was transferred to a platinum crucible and heated to 1,000-1,600° C. in a Muffle furnace. 20 minutes later, a melted glass mixture was stirred with a quartz rod and heated again for 10 minutes. A certain quantity of Na₂SO₄ or K₂CO₃ was taken, placed in a quartz grinder and fully ground for 10 minutes. The melted glass mixture was poured, stirred with a quartz rod, heated for 5 minutes, then taken out and slowly poured into water for water quenching. Then, a prepared glass material was ground into a glass powder with an irregular shape and a particle size of 1-100 μm with a ball mill. The glass powder was subjected to further particle size sorting through an ultrasonic vibrating screen (vibrating for 20 minutes) equipped with a 10 μm filter screen and a 40 μm filter screen, and a glass powder on the 10 μm filter screen was collected. The glass powder was transferred into a flame spheroidization furnace with nitrogen as a carrier gas and an oxygen-acetylene flame as a spheroidization flame (the flame has a temperature of greater than 3,000° C., and the furnace has a temperature of about 1,600-1,800° C.). The glass powder with an irregular shape was melted in a spherical shape, and a foaming agent inside was decomposed to release gases so as to form cavities. Microspheres were collected with a collector at the bottom of the flame spheroidization furnace. The obtained microspheres were first sieved through an ultrasonic vibrating sieve (vibrating for 20 minutes) equipped with a 20 μm filter and a 50 μm filter, and then microspheres on the 20 μm filter were collected.

TABLE 1
Molar percentages of various components of radioactive glass
microspheres with sample numbers S101-S104 and H101 to H111

| | Y₂O₃/ | Al₂O₃/ | SiO₂/ | B₂O₃/ | Na₂SO₄/ | K₂CO₃/ |
|---|---|---|---|---|---|---|
| | molar | molar | molar | molar | molar | molar |
| Sample | % | % | % | % | % | % |
| S101 | 18 | 18 | 64 | 0 | 0 | 0 |
| S102 | 19 | 18 | 63 | 0 | 0 | 0 |
| S103 | 20 | 22 | 58 | 0 | 0 | 0 |
| S104 | 20 | 22 | 48 | 10 | 0 | 0 |
| H101 | 18 | 18 | 63 | 0 | 1 | 0 |
| H102 | 18 | 18 | 62 | 0 | 2 | 0 |
| H103 | 18 | 18 | 61 | 0 | 3 | 0 |
| H104 | 18 | 18 | 63 | 0 | 0 | 1 |
| H105 | 18 | 18 | 62 | 0 | 0 | 2 |
| H106 | 18 | 18 | 61 | 0 | 0 | 3 |
| H107 | 18 | 18 | 62 | 0 | 2 | 0 |
| H108 | 19 | 22 | 57 | 0 | 2 | 0 |
| H109 | 20 | 22 | 46 | 10 | 2 | 0 |
| H110 | 20 | 22 | 49 | 7 | 2 | 0 |
| H111 | 20 | 22 | 51 | 5 | 2 | 0 |

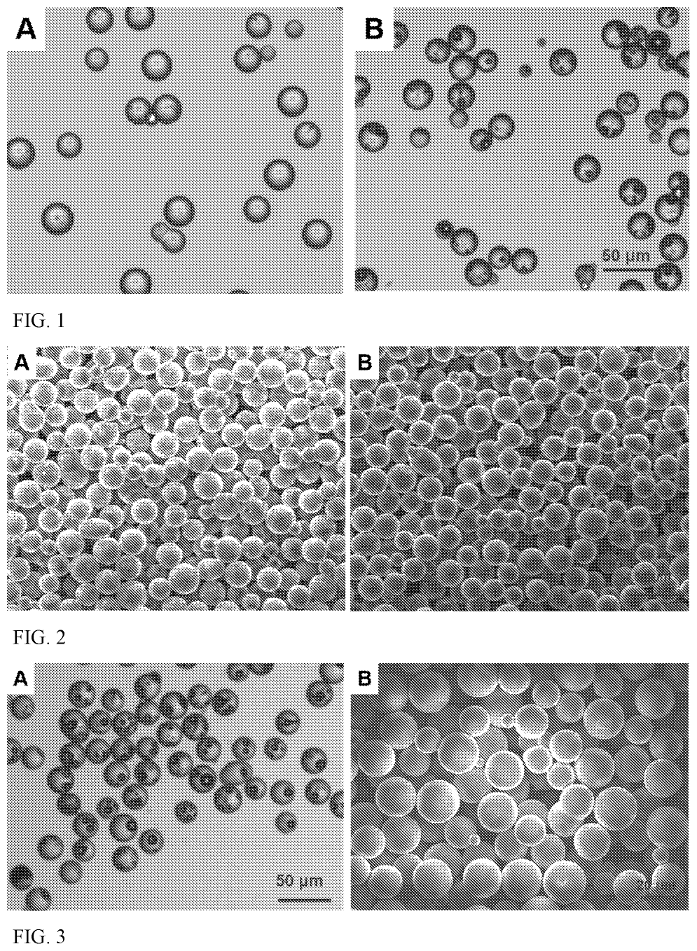

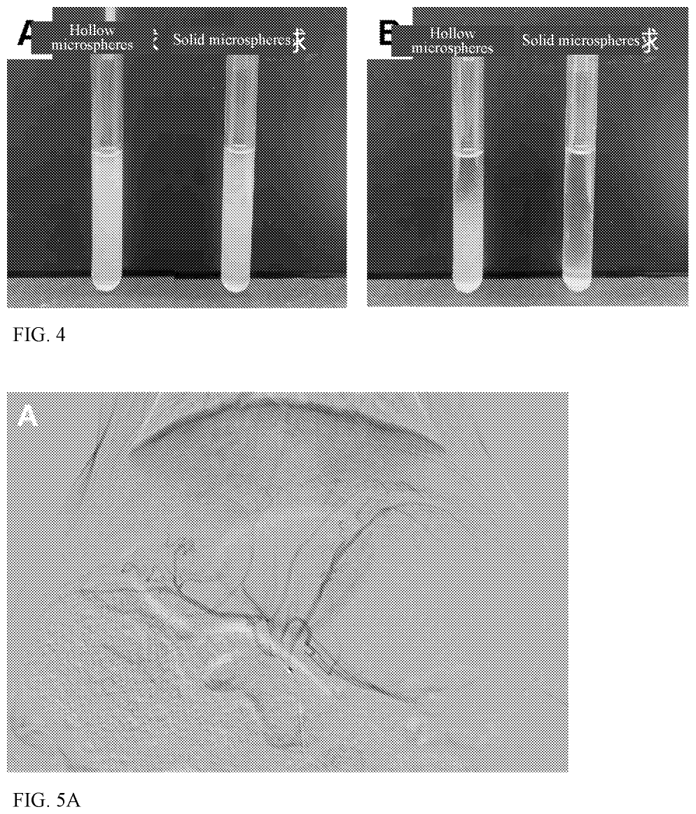

The glass transition temperature of 14 kinds of glass microsphere samples in Table 1 was measured by a differential scanning calorimeter (DSC3, Mettler Toledo) in Example 1 of the present invention, and specific results are as shown in Table 2. In addition, the density of the glass microsphere samples in Table 1 was measured, and the results are as shown in Table 2. The signal intensity of corresponding nuclides in the glass microsphere samples-in Table 1 was measured by inductively coupled plasma-mass spectrometry (ICP-quadrupole-MS, Varian 810-MS, USA) and compared with standard curves of the corresponding nuclides to determine the final nuclide content. Nuclide loading rate (%)=mass of nuclide/total mass of microspheres for embolization×100%, and the results are as shown in Table 2. 100 mg of the samples in Example 1 were subjected to neutron irradiation for 6 hours (the neutron flux is about 5×10¹³ neutrons/cm²·s) respectively, and the radiation dose of each sample was measured by a dose calibrator (Atomlab 100). The S104 and H109 microspheres prepared were suspended in a 0.1% Tween 20 solution and then dropped into a hemocytometer, and optical photographs of the microspheres were taken with a microscope. As shown in FIG. 1, the left figure in FIG. 1 is a microscope photograph of solid glass microspheres, and the right figure is a microscope photograph of hollow glass microspheres. A scanning electron microscope image in FIG. 2 shows that the hollow microspheres have smooth and complete surfaces, and cavities are only present in the glass microspheres, which have no impact on flowing of the glass microspheres in catheters or blood vessels and have no impact on the integrity of embolization.

TABLE 2
Density, glass transition temperature, nuclide loading
rate and radiation dose of glass microsphere samples

| | | | Nuclide | |
|---|---|---|---|---|
| | Density/ | T_{g}/° | loading | Radiation |
| Sample | g/cm³ | C. | rate/wt % | dose/MBq |
| S101 | 3.56 | 882 | 33.7% | 2480 |
| S102 | 3.61 | 895 | 35.6% | 2500 |
| S103 | 3.67 | 905 | 35.8% | 2510 |
| S104 | 3.59 | 893 | 34.2% | 2490 |
| H101 | 2.13 | 885 | 33.5% | 3190 |
| H102 | 1.96 | 887 | 34.7% | 3280 |
| H103 | 1.73 | 884 | 34.1% | 3460 |
| H104 | 1.93 | 882 | 33.5% | 3450 |
| H105 | 1.66 | 885 | 34.7% | 3410 |
| H106 | 1.41 | 892 | 33.9% | 3470 |
| H107 | 2.28 | 887 | 34.1% | 3470 |
| H108 | 2.34 | 899 | 34.2% | 3400 |
| H109 | 1.76 | 857 | 36.4% | 3800 |
| H110 | 1.88 | 864 | 36.0% | 3890 |
| H111 | 1.93 | 873 | 36.2% | 3860 |

It can be seen from Table 2 that compared with solid glass microspheres (S101-S104), the density of glass microspheres (H101-H111) added with a foaming agent is significantly decreased and is generally about 1.4-2.3 g/cm³, which is much lower than that of the solid microspheres (greater than 3.5 g/cm³). The reason is that after a foaming agent is added, the overall glass microspheres are more loose in texture and have more cavities therein, and the density is greatly decreased in comparison with the solid glass microspheres, which is more similar to the density of blood. Thus, the hollow glass microspheres are more suitable for use as radioactive glass microspheres for embolization. The hollow glass microspheres are not quickly sedimentated in liver blood vessels after injection, but can be uniformly and stably distributed in the liver blood vessels to achieve better distribution and deposition effects and a better embolization effect, thereby significantly improving the therapeutic effect.

Compared with the solid glass microspheres (S101-S104), the glass microspheres (H101-H111) added with a foaming agent have little change in glass transition temperature, a slightly increased nuclide loading rate and an obviously different radiation dose, which is increased from about 2,500 MBq to about 3,600 MBq. The reason may be that the glass microspheres (H101-H111) added with a foaming agent are loose in texture and contain many bubbles and cavities therein. After neutron activation of a nuclide in the glass microspheres, a β-ray emitted by yttrium-90 is more likely to radiate from a loose glass matrix with cavities and can produce a higher radiation dose and kill surrounding tumor cells more effectively.

Through comparison of glass matrix formulas of the two kinds of glass microspheres, it can be seen that in the glass microspheres (H101-H111) added with a foaming agent, when the glass matrix contains B₂O₃, the nuclide loading rate is high, and the radiation dose is obviously higher. After analysis of the microspheres, the reason may be that as the glass microspheres containing B₂O₃ have better properties, convenience is provided for foaming of the foaming agent to further improve the texture and fix more nuclide oxides, and radiation of a β-ray is more facilitated.

In addition, two different foaming agents including Na₂SO₄ or K₂CO₃ have no obviously different foaming effects in preparation of the glass microspheres. With increase of the-amount of the foaming agent Na₂SO₄ or K₂CO₃, the density of the prepared glass microspheres is further decreased, but the nuclide loading rate and the radiation dose are not continuously increased. Therefore, the added amount of Na₂SO₄ or K₂CO₃ is preferably 2 molar %.

Therefore, in order to improve comprehensive performance of radioactive glass microspheres for embolization, a foaming agent is required to be added for preparing glass microspheres with cavities. B₂O₃ is required to be added in a glass matrix formula, and 2 molar % Na₂SO₄ or K₂CO₃ can be used as a foaming agent.

### Example 2 Density Screening of Radioactive Glass Microspheres for Embolization

In this example, in order to further reduce density differences between microspheres, density screening is performed on the microspheres. The microspheres are placed in a solvent with a specific density for centrifugal separation, a precipitated part includes microspheres with a density greater than that of the solvent, and a supernatant part is the part with a density less than that of the solvent. Specific operations are as follows.

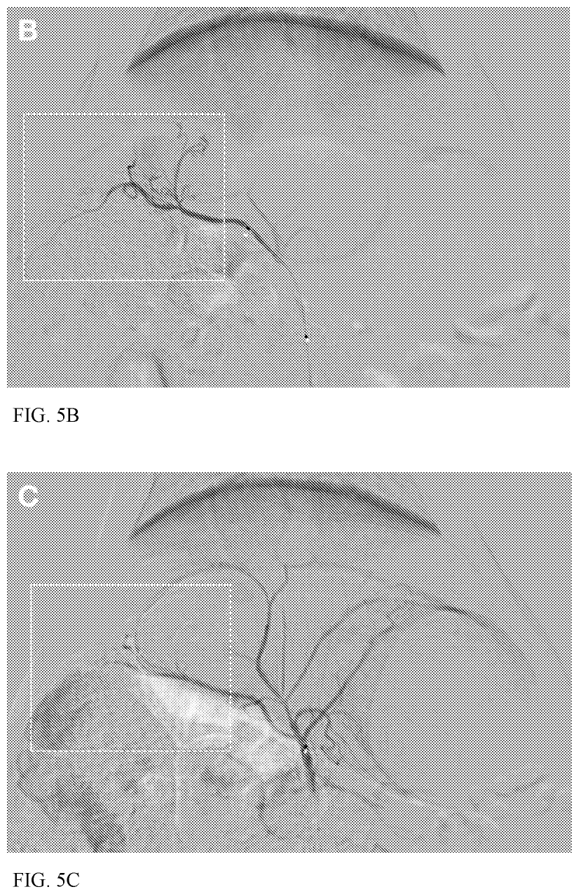

10 g of H102 microspheres (prepared in Example 1) were weighed, added into 40 mL of 1,2-dibromoethane (with a density of 2.17 g/cm³) and shaken for 30 seconds for uniform mixing, followed centrifugation for 10 minutes (at 5,000×g), and 15 mL of an emulsion on the upper layer was collected. Then, the microspheres were added into 35 mL of 1,2-dibromoethane and shaken for 30 seconds for uniform mixing, followed by centrifugation for two times for 10 minutes (at 5,000×g), and 10 mL of an emulsion on the upper layer was collected. The obtained microspheres were placed in an oven for drying at 60° C. for more than 2 hours. The dried microspheres were added into 40 mL of a 3 M fluorinated liquid FC40 (with a density of 1.85 g/cm³) and shaken for 30 seconds for uniform mixing, followed by centrifugation for 10 minutes (at 5,000 g), and 35 mL of a solution on the upper layer was removed. Then, the microspheres were added into 35 mL of a 3 M fluorinated liquid FC40 and shaken for 30 seconds for uniform mixing, followed by centrifugation for 10 minutes (at 5,000×g), and 35 mL of a solution on the upper layer was removed. The microspheres at the bottom were placed in an oven for drying at 60° C. for more than 2 hours to make the residual FC40 completely volatilized. The obtained microspheres are as shown in FIG. 3, which have a density of less than 2.17 g/cm³ and greater than 1.85 g/cm³.

### Example 3 Comparison in Sedimentation Velocity

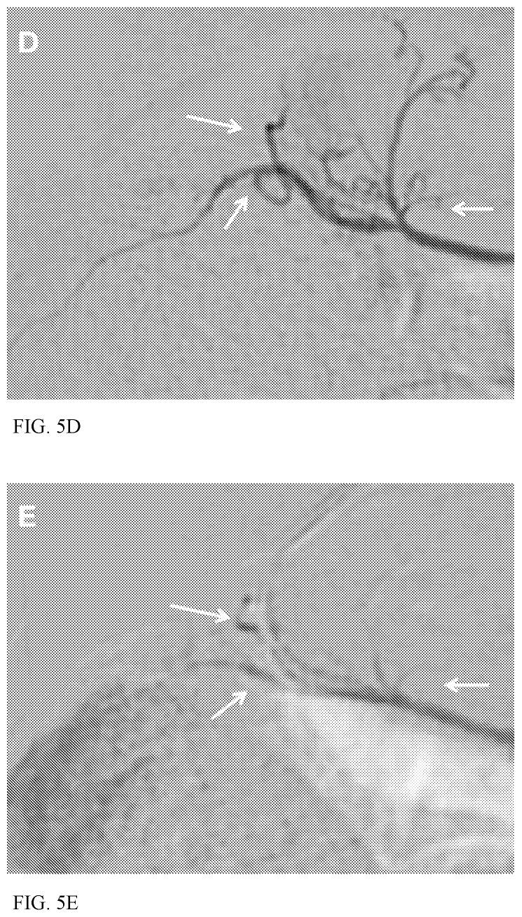

In order to evaluate the sedimentation velocity of hollow microspheres, 40 mg of solid microspheres (S101) and hollow microspheres prepared in Example 2 were weighed and added into 5 mL of PBS for uniform mixing, followed by standing for 1 minute, respectively, and natural sedimentation photographs of the microspheres in PBS were taken, as shown in FIG. 4.

It can be seen from FIG. 4 that 1 minute later, the solid microspheres are basically sedimentated to the bottom completely due to a high density, while the hollow microspheres are only sedimentated to ⅓ of a liquid column height.

Thus, it can be seen that due to a high density, existing solid glass microspheres are quickly sedimentated to every corner of the bottoms of blood vessels after each injection and are difficult to be accurately located for achieving a good vascular embolization effect. However, the glass microspheres with cavities, which are prepared by foaming with a foaming agent and provided by the present invention, are not immediately sedimentated to the bottoms of blood vessels after injection, but can be uniformly distributed on upper sides, lower sides, left sides and right sides of vertical interfaces in the blood vessels and accurately accumulate in sites requiring vascular embolization until vascular embolization is realized, so as to achieve a better therapeutic effect.

### Example 4 Comparison with Glass Microspheres on the Market

10 mg of the microspheres obtained in Example 2 were taken, suspended in 500 μL of a 0.1% Tween 20 solution and dropped into a hemocytometer, and an optical photograph of the microspheres was taken with a microscope. The microspheres have one or more cavity structures. The particle size of the microspheres was analyzed and measured by image processing software Image J. The microspheres have an average diameter of 26.8 μm. The nuclide loading rate of the microspheres for embolization was measured by inductively coupled plasma-mass spectrometry. 5 mg of the microspheres for embolization prepared in Example 2 were weighed, and the signal strength of corresponding nuclides was measured by inductively coupled plasma-mass spectrometry (ICP-quadrupole-MS, Varian 810-MS, USA) and compared with standard curves of the corresponding nuclides to determine the final nuclide content. Nuclide loading rate (%)=mass of nuclides/total mass of microspheres for embolization×100%. Through calculation and analysis, the nuclide loading rate is 34.8%. Test results are as shown in Table 3.

TABLE 3
Comparison of parameters between microspheres for embolization
with cavities prepared in Example 2 and TheraSphere ® solid
microspheres for embolization

| | Diameter (μm) | Density (g/cm³) | Loading rate (%) |
|---|---|---|---|
| Example 2 | 26.8 | 1.85-2.07 | 34.8 |
| TheraSphere | 25 | 3.6 | 31.5 |

It can be seen from Table 3 that compared with TheraSphere® glass microspheres, the hollow glass microspheres for embolization of the present invention have the advantages that as cavities are formed on the glass microspheres, the density of the glass microspheres for embolization is effectively decreased, and the hollow glass microspheres have good distribution and deposition effects during injection. Moreover, it can also be ensured that the glass microspheres provided by the present invention have a high nuclide loading rate.

### Example 5 Animal Experiment on Hollow Microspheres of the Present Invention and Solid Microspheres

### 1. Materials and Reagents

A Sumianxin II injection for animals, pentobarbital sodium, an animal fixing frame, an animal dissection plate, a 1 mL syringe, a 5 mL syringe, a 10 mL syringe, an animal shaver, a surgical blade, surgical vessel forceps, surgical scissors, a surgical suture needle, tweezers, a needle holder, a 4-0 surgical suture, an arterial puncture needle, a 0.038 inch radifocus guide wire, a 2% lidocaine injection, a 1.7F Ev3 microcatheter with a guide wire, microspheres for embolization (hollow microspheres prepared by the present invention and solid microspheres on the market: TheraSphere® glass microspheres), ioversol, a disposable surgical drape, iodophor, sterile surgical gloves, heparin sodium and normal saline were used.

### 2. Anesthesia

A rabbit was fixed onto an animal fixing frame, 0.2 mL of a Sumianxin II injection for animals was intramuscularly injected into a leg, and then 0.8 mL/kg of a 3% pentobarbital solution was intravenously injected into the margin of an ear.

### 3. Femoral Artery Puncture and Cannulation

(1) After the rabbit was anesthetized, four limbs were opened and fixed onto an animal dissection plate to fully expose an inner side surface of one hind limb, hair on an inner side of the leg was shaved with an animal shaver, the inner side was disinfected with iodophor for 3 times, and then a disposable surgical drape was laid.
(2) After subcutaneous layered injection of a 2% lidocaine hydrochloride injection at a site where pulsation of a femoral artery was touched, an epidermal layer and a muscle fascia layer were cut layer by layer with a surgical blade in a pulsation direction of the femoral artery, and a section of the femoral artery was bluntly separated layer by layer with vessel forceps at a site near a vagina vasorum of the femoral artery.
(3) A distal end of the femoral artery was ligated with a suture to make a proximal end fully dilated, an arterial puncture needle was gently punctured into the proximal end in the direction of the femoral artery to send a puncture needle cannula sheath into the femoral artery while a needle core was removed. Bright red arterial blood spilling after the needle core was removed indicated that arterial cannulation was successful. Then, a radifocus guide wire was quickly sent into the femoral artery. The guide wire was observed to enter the aorta by fluoroscopy, which once again confirmed that femoral artery puncture and cannulation were successful. The cannula sheath was fixed in the femoral artery with a suture.

### 4. Hepatic Arteriography

The radifocus guide wire was removed, and a 1.7F Ev3 microcatheter and a micro-guide wire were sent. An abdominal artery near a T12 centrum was found, and an ioversol contrast agent diluted with normal saline containing heparin sodium at a ratio of 1:1 was injected to develop a common hepatic artery and a proper hepatic artery. After the 1.7F Ev3 microcatheter was cannulated into the common hepatic artery, digital subtraction angiography was performed to fully display blood supply conditions of hepatic arteries.

### 5. Embolization of a Hepatic Artery

In the case of angiography, equal quantities of solid microspheres and hollow microspheres for embolization were fully suspended in diluted ioversol and slowly injected into a target artery for embolization. Flowing conditions of the contrast agent in the artery branch were carefully observed, and embolization was stopped when reverse flowing occurred. Hepatic arteriography was performed again to determine embolization conditions of blood vessels.

### 6. Completion of Surgery

The microcatheter and the micro-guide wire were pulled out, the cannula sheath was removed, the proximal end of the femoral artery was quickly ligated, the muscle layer and the epidermal layer of the thigh of the rabbit were sutured layer by layer with a disposable suture needle and a suture and disinfected, and surgery was completed.

### 7. Results and Analysis

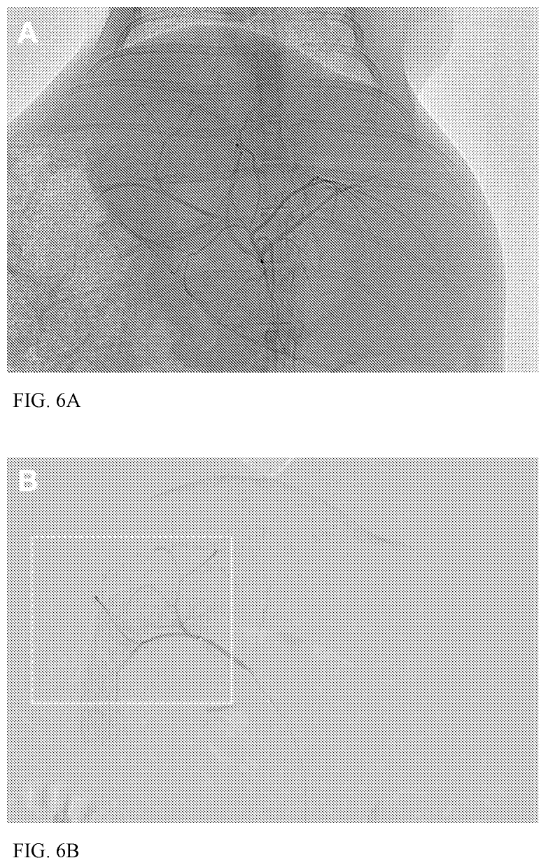

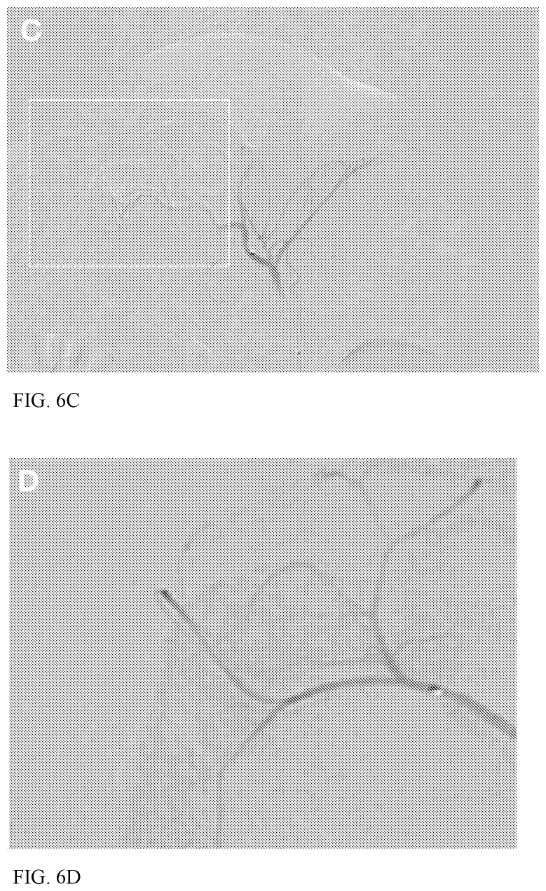

FIG. 5A to FIG. 5E are images showing embolization of solid glass microspheres, in which FIG. 5A is an angiography image of overall blood vessels before embolization, and it can be seen that the blood vessels are complete and clear; FIG. 5B is an enlarged image of a selected embolization area of solid glass microspheres, and it can be seen that blood vessels in the selected embolization area are complete and clear; FIG. 5C is a locally enlarged image of a selected embolization area; FIG. 5D is an angiography image of solid glass microspheres in a selected embolization area; and FIG. 5E is a locally enlarged image of an angiography image of solid glass microspheres in a selected embolization area. Through comparison between FIG. 5E and FIG. 5C, it can be clearly seen that when the solid glass microspheres are used for vascular embolization, the embolization is not complete, white angiography only appears locally, and blood still flows in some blood vessels, indicating that the solid microspheres cannot have a good embolization effect on blood vessels. The reason may be that due to a large density, the solid microspheres are difficult to flow, thus increasing the possibility of incomplete therapy caused by too early sedimentation in blood vessels. In addition, due to a relatively high density, the probability that the microspheres fall back into a gastroduodenal artery is increased, resulting in possible abdominal pain, vomiting and even gastroduodenal perforation.

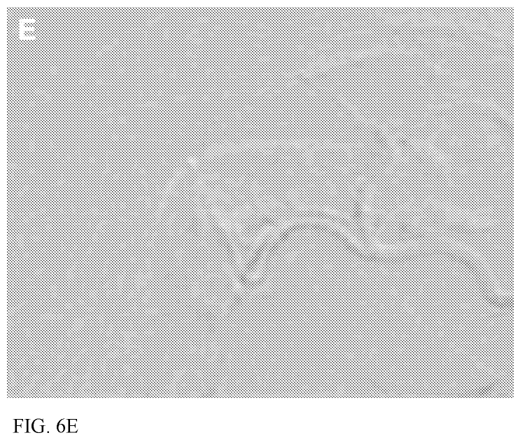

In contrast, when the hollow glass microspheres of the present invention are used for vascular embolization in an embolization area, basically all blood vessels are filled with the hollow glass microspheres. Through comparison between FIG. 6B and FIG. 6C (or comparison between locally enlarged images FIG. 6D and FIG. 6E) as well as FIG. 6A, which is an image of blood vessels without embolization, it can be seen that all blood vessels are filled with the hollow microspheres, and the microspheres are uniformly distributed. When tumor tissues near the blood vessels are found, the hollow microspheres can effectively kill the tumor cells during follow-up radiation therapy. Moreover, due to a relatively lower density, the probability that the microspheres fall back into the gastroduodenal artery is greatly decreased, and complications obtained after embolization surgery are reduced to a maximum extent.

All patents and publications referred to in the specification of the present invention indicate that these patents and publications are open technologies in the art and can be used in the present invention. All the patents and publications cited herein are also listed in references as each publication is specifically cited separately. The present invention described herein may be realized under the conditions of absence of any one or more elements and presence of one or more limitations, and the limitations are not specified herein. For example, in each instance, the terms “contain”, “substantially composed of . . . ” and “composed of . . . ” can be replaced by either one of the remaining 2 terms. The so-called “one” herein only means “one” and can also mean 2 or above without excluding the meaning of only including one. The terms and expressions used herein are description manners, and the present invention is not limited thereto. Any intentions indicating that the terms and interpretations described herein exclude any equivalent features are also unavailable herein. However, it is to be understood that any suitable changes or modifications can be made within the scope of the present invention and the claims. It is to be understood that the examples described in the present invention are preferred examples and characteristics, some alternations and changes can be made by any person of general skill in the art according to the essence of descriptions of the present invention, and all these alternations and changes are also considered as falling within the scope of the present invention and the scope limited by the independent claims and the dependent claims.

1. Radioactive glass microspheres for embolization, wherein the glass microspheres comprise glass microsphere bodies and cavities formed in the glass microsphere bodies, and the glass microsphere bodies contain a nuclide oxide; and the glass microspheres have a density of not higher than 2.3 g/cm³.

2. The glass microspheres according to claim 1, wherein the nuclide oxide is any one of Y₂O₃, Lu₂O₃, Ho₂O₃ or P₂O₅.

3. The glass microspheres according to claim 2, wherein the glass microsphere bodies further contain any one of Al₂O₃, SiO₂ and B₂O₃.

4. The glass microspheres according to claim 3, wherein the glass microspheres have a density of 1.4-2.3 g/cm³.

5. The glass microspheres according to claim 4, wherein the glass microspheres have a nuclide loading rate of 15-40 wt %.

6. The glass microspheres according to claim 5, wherein the glass microspheres have a density of 1.6-1.9 g/cm³ and a nuclide loading rate of 33-36 wt %.

7. The glass microspheres according to claim 6, wherein the glass microspheres contain, by molar percentage, 0-40% of Al₂O₃, 20-80% of SiO₂, 0-20% of B₂O₃ and 10-30% of a nuclide oxide, and have a particle size of 10-100 μm.

8. The glass microspheres according to claim 7, wherein the glass microspheres contain, by molar percentage, 18-22% of Al₂O₃, 45-63% of SiO₂ and 0-10% of B₂O₃.

9. A method for preparing the radioactive glass microspheres for embolization according to claim 1, comprising the following steps:
(1) preparing glass microsphere bodies, mixing the glass microsphere bodies with a foaming agent to obtain a mixture, and melting the mixture by heating to obtain a glass matrix;
(2) cooling the glass matrix, followed by curing to form a glass block, and then grinding the glass block to obtain glass particles; and
(3) melting the glass particles by heating to obtain glass microspheres, and decomposing the foaming agent in the glass microspheres to generate gases so as to form cavities in the glass microspheres.

10. The method according to claim 9, wherein the glass microsphere bodies contain any one of Al₂O₃, SiO₂ and B₂O₃, and a nuclide oxide.

11. The method according to claim 10, wherein the nuclide oxide is any one of Y₂O₃, Lu₂O₃, Ho₂O₃ or P₂O₅.

12. The method according to claim 7, wherein the foaming agent in step (1) comprises one or more of Na₂SO₄, MgSO₄, Na₂CO₃, CaSO₄, K₂CO₃, Li₂CO₃ and SrCO₃.

13. The method according to claim 7, wherein during the melting by heating in step (1), the heating is performed at a temperature of 1,000-1,600° C.; and during the melting by heating in step (3), the heating is performed at a temperature of 1,600-1,800° C.

14. The method according to claim 9, wherein the method further comprises a step (4): screening glass microspheres with a suitable particle size of 10-100 μm and a density of 1.4-2.3 g/cm³.

15. A method for preparing the radioactive glass microspheres for embolization according to claim 1, comprising the following steps:
(a) preparing glass microsphere bodies, and melting the glass microsphere bodies by heating to obtain a glass matrix;
(b) cooling the glass matrix, followed by curing to form a glass block, adding a foaming agent for mixing, and then performing grinding to obtain glass particles so as to make the foaming agent adsorbed on the surfaces of the glass particles; and
(c) melting the glass particles by heating, and decomposing the adsorbed foaming agent to generate gases so as to form cavities in the glass microspheres.

16. The method according to claim 15, wherein the glass microsphere bodies contain any one of Al₂O₃, SiO₂ and B₂O₃, and a nuclide oxide.

17. The method according to claim 16, wherein the nuclide oxide is any one of Y₂O₃, Lu₂O₃, HO₂O₃ or P₂O₅.

18. The method according to claim 15, wherein the foaming agent in step (b) comprises any one or more of polyethylene glycol and polyvinyl alcohol.

19. The method according to claim 15, wherein during the melting by heating in step (a), the heating is performed at a temperature of 1,000-1,600° C.; and during the melting by heating in step (c), the heating is performed at a temperature of 1,600-1,800° C.

20. The method according to claim 15, wherein the method further comprises a step (d): screening glass microspheres with a suitable particle size of 10-100 μm and a density of 1.4-2.3 g/cm³.
